(19) United States
(12) Patent Application Publication
(10) Pub. No.: US 2007/0084470 A1
(43) Pub. Date: Apr. 19, 2007

(54) Anti-snoring apparatus

(76) Inventor: John Thomas Sarazen, New Providence, NJ (US)

Correspondence Address: Mr. John T. Sarazen, 205 Sagamore Drive, New Providence, NJ, 07974, US

(21) Appl. No.: 11/251,503

(22) Filed: Oct. 17, 2005

(51) Int. Cl. A61F 5/56

(52) U.S. Cl. 128/848

(57) ABSTRACT

The present invention provides an improved method and apparatus for treatment of snoring in adults. The method provides the steps for snoring reduction or elimination through jaw and mouth support, acupressure, magnetic therapy, and optional electronically controlled 9-volt electro-magnetic current applied to and around certain acupuncture points on the wearer's head. The apparatus is a device that is designed to be worn by a snorer while sleeping to aid in reducing or eliminating snoring in adults. In a preferred form of the invention, the user wears an apparatus on the head during sleep which is comprised of two major parts: two composite ear pads, an adjustable elastic head, jaw, chin and lower mouth support system; this apparatus reduces or eliminates snoring in many adults. As will be apparent to those skilled in the art, various modifications of the method and apparatus described above can be made without departing from the spirit of the invention, the scope of which is to be construed in light of the claims made herein.

### CROSS-REFERENCE TO RELATED APPLICATIONS

None

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### BACKGROUND OF THE INVENTION

The invention relates to the treatment of non-apnea snoring in adult humans, and more particularly to a method and apparatus for the treatment of snoring which is not directly caused by obstructive sleep apnea, a serious and potentially life-threatening sleep disorder, or other sleep-related disorders.
1. Snoring affects an estimated 65% of the adult U.S. population, according to leading medical organizations such as the American Medical Association (AMA), American Sleep Apnea Association, The U.S. Centers for Disease Control (CDC), the Mayo Clinic and many others; and as many as 75% of adults ages 55 and older may snore one or more times a night. While chronic, loud snoring is often a symptom of obstructive sleep apnea (OSA), not all snorers suffer from this potentially life-threatening sleep disorder. The US Food and Drug Administration (FDA) has stated that about ⅓ of adult snorers, mostly those who are overweight, may be at risk of OSA. As many Americans are generally becoming more and more overweight, according to CDC and AMA research, the prevalence of adult snoring will likely increase.
2. There are two widely accepted causes of adult snoring: oral or mouth-caused, and nasal. According to accepted medical practice, as much as 85% of nocturnal, non-apnea snoring is caused by open-mouthed (oral) breathing during sleep, and is largely due to vibration of the fleshy portions of the pharynx, including the uvula. In a number of academic studies in the United States and abroad, sometimes chronic snoring (most probably related to OSA) in adults has been linked to increased risks of heart disease, strokes, diabetes, arthritis and other diseases, including sleep apnea, a potentially life-threatening sleep disorder.

There have also been a small number of European, Canadian, and Israeli sleep studies that suggest that OSA may possibly be causally linked to snoring such that chronic, loud and disruptive snoring may actually trigger the onset of some forms of OSA, and not the other way—that OSA causes chronic snoring. This interpretation is not widely accepted in the United States, but it is based on some clinical data that suggests that OSA may be due to other factors such as overweight and obesity and may be an outcome of uncontrolled snoring in otherwise healthy adults. Even in snorers who do not have OSA or other sleep disorders, loud and frequent snoring can significantly disrupt the snorer's sleep, as well as that of persons in the immediate vicinity (spouse, children, even pets), causing chronic tiredness, increased susceptibility to illness, and reduced job performance and productivity. Snoring and related sleep disorders are estimated by various expert sources to cost the U.S. economy more than $3 billion in lost productivity each year.
3. Snoring can be loud, reaching more than 82 db, about the loudness of a gasoline lawnmower running at full speed, affecting not only the snorer, but also family members and pets and even neighbors. In some instances, chronic snoring may be linked to a serious sleep disorder, sleep apnea, the periodic cessation of breathing during sleep. Sleep apnea requires medical diagnosis and treatment; this method and apparatus is not intended to treat obstructive sleep apnea or any disease.
4. Although considerable research into the causes and treatment of snoring has been done over many decades, relatively little progress has been made towards successful treatment of this problem that affect so many adults, especially those over the age of 45.
5. Many leading treatments today focus on invasive surgery with its attendant health risks, or the use of a wide range of devices, including mouthguards, nose and chin tapes, oral inserts and a variety of headbands designed to keep the mouth closed during sleep. Temporary reduction or cessation of non-apnea snoring may be achieved in many cases through the use of non-intrusive devices that may help keep the mouth closed during sleep.

Other approaches to the reduction of adult non-apnea snoring include surgery, biofeedback, oral mouthpieces and dental appliances, and other means. While the method and apparatus described in this patent application can be helpful in reducing adult non-apnea snoring in many instances, it does not work for everyone and should be used by adults who do not have sleep apnea or other snoring-related sleep disorders; disorders which can only be diagnosed and treated by properly licensed medical professionals.

### DESCRIPTION OF THE PRIOR ART

Various approaches have been proposed for the treatment of chronic snoring. These include approximately 1,125 U.S. patents issued since 1790 in the subjects of snoring, magnetic therapy, and acupressure. U.S. and international patents issued with some relevance to the above claim include the following U.S. patents:

i) Head strap assembly for reducing snoring activity (1997 U.S. Pat. No. 5,687,743)

ii) Head cover and chinstrap for treating sleep apnea (1994 U.S. Pat. No. 5,361,416)

iii) Head strap assembly for reducing snoring (1950 U.S. Pat. No. 2,507,617)

iv) Device to prevent mouth breathing and snoring (1935 U.S. Pat. No. 1,990,411)

v) Anti-mouth breathing device (1920 U.S. Pat. No. 1,339,865)

vi) Cervical appliance to ameliorate sleep apneas (1987 U.S. Pat. No. 4,700,697)

vii) Facial contouring mask (1987 U.S. Pat. No. 4,658,811)

viii) Acupressure treatment device (2001 U.S. Pat. No. 6,261,306)

ix) Therapeutic head cradle (2001 U.S. Pat. No. 6,182,313)

x) Acupressure treatment device (2000 U.S. Pat. No. 6,030,408)

xi) Apparatus for magnetic therapy of sinus conditions (2000 U.S. Pat. No. 6,053,859)

xii) Mask for applying a magnetic flux field to facial skin (1998, U.S. Pat. No. 5,738,624)

xiii) Magnetic medical treatment device (1975 U.S. Pat. No. 3,921,620)

xiv) Magnetic therapeutic device (1989 U.S. Pat. No. 4,798,194)

xv) Flexible magnet (1997 U.S. Pat. No. 5,621,369)

xvi) Method of alleviating pain using a low density magnetic field (1998 U.S. Pat. No. 5,807,233)

xvii) Modular magnetic therapy device (1999 U.S. Pat. No. 5,993,375)

xviii) Cold therapy pad with magnetotherapy insert (2000 U.S. Pat. No. 6,129,659)

xix) Magnetotherapeutic mask (2004 U.S. Pat. No. 6,692,427)

xx) Treatment of afflictions, aliments and diseases (2002 U.S. Pat. No. 6,379,295)

xxi) Magnetic therapy patch (2002 U.S. Pat. No. 6,344,021)

xxii) Flexible magnetic insole (2001 U.S. Pat. No. 6,322,491)

xxiii) Apparatus and method for the treatment of disorders of tissue and/or the joints (2002 U.S. Pat. No. 6,447,440)

xxiv) Articulated electromagnet (1890 U.S. Pat. No. 435,343)

xxv) Foot and ankle reflexology bands (2003 U.S. Pat. No. 6,527,792)

xxvi) Deep heat magnetic wrap for joints and tissues (2003 U.S. Pat. No. 6,652,446)

xxvii) Therapeutic clothing and method of use (2004 U.S. Pat. No. 6,763,525)

xxviii) Headband for treatment of headaches (2003 U.S. Pat. No. 6,554,787)

xxix) Acupressure treatment device (2001 U.S. Pat. No. 6,261,306)

xxx) Electrical pulse acupressure system (1982 U.S. Pat. No. 319,584)

### BRIEF DESCRIPTION OF THE DRAWINGS

In drawings which illustrate an embodiment of the invention:

FIG. 1 is a side perspective view of the device of the invention in use on a human head:

(1) non-woven fabric sleep cap

(2) embedded earpad system

(3) fabric “wings” that provide gentle support for the mouth, jaw and chin.

FIG. 2 is an exploded side view of the device of the invention, showing the design of the earpad:

(4) non-woven fabric cover,

(5) plastic substrate/circuit and connectors for the optional electronic control system,

(6) acupressure/magnetic earpad system.

FIG. 3—Top View of Earpad System—line drawing of earpad design showing the engineered shape, designed to optimize physical and magnetic stimulation of certain acupuncture and acupressure points on and around the ear.

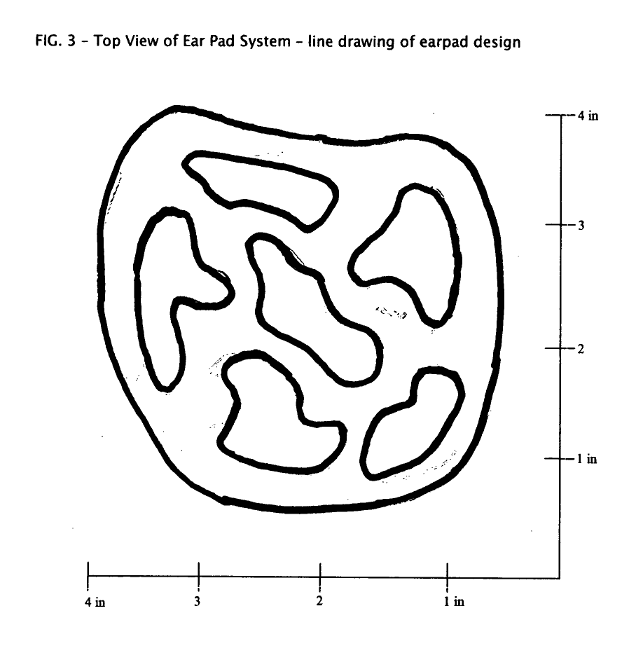

FIG. 4—Schematic View of magnetic “hotspots” created by earpad, generated by spraying very fine iron powder over earpad. Shows areas where magnetic field created by earpad generates “hotspots” that may stimulate acupuncture and acupressure points on and around the ear.

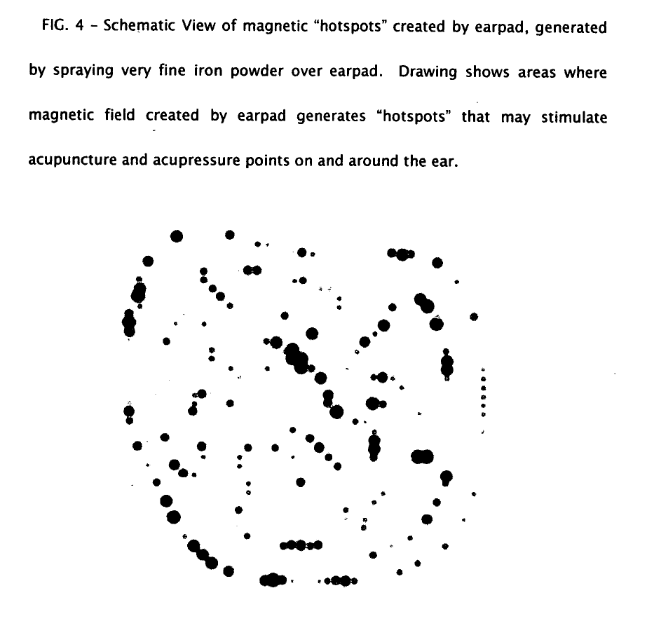

### DETAILED DESCRIPTION OF THE INVENTION

The present invention provides an improved method and apparatus for the treatment of snoring in adults. The method provides for integrated:
1. Bio-mechanical support for the jaw, chin, and mouth engineered to keep the mouth closed during sleep;
2. Intermittent and persistent physical stimulus and pressure, which can be applied to certain acupuncture and acupressure points as documented in the World Health Organization's (WHO) Medical Library Proposed Standards for acupuncture and acupressure. This activity by the WHO is in response to the broad worldwide use of acupuncture and acupressure as one of many complementary treatment approaches. More information on the WHO Program for alternative healthcare may be found at:

(http://www.who.int/medicines/library/trm/acupuncture/proposedstandacup.shtml);
3. Magnetic therapy—which can be applied in both a diffuse manner to certain acupuncture and via specified acupressure “points” through the magnetic substrate in the device; and also in a more focused manner through additional optional embedded magnetic materials that can be molded into the ear pads, and also into the head and mouth support component parts of the device as needed.
4. Optional pulsed and/or persistent electrical stimulation of certain acupuncture and acupressure “points” through the application of a very low-voltage electro-magnetic field, delivered by an optional 9-volt battery powered electronic controller and power system. This feature may provide additional benefits to help reduce snoring and aid in relaxation.
5. In a preferred form of the invention, the user wears the apparatus like a sleep cap with two “wings” supporting the chin, which keeps the mouth closed during sleep. Prior to going to sleep, the user can adjust the device to help keep the mouth closed during sleep and position the ear pads for comfort. The user is able to sleep comfortably on his/her back or side through the night with greatly reduced or eliminated snoring.
6. The acupressure, magnetic therapy and optional electro-magnetic field features of the apparatus help reduce stress, induce sleep, and aid in lessening the tendency of users toward snoring through the mouth, thus reducing or eliminating the most common source of non-apnea caused snoring—open mouth breathing.

### DETAILED DESCRIPTION OF A PREFERRED EMBODIMENT

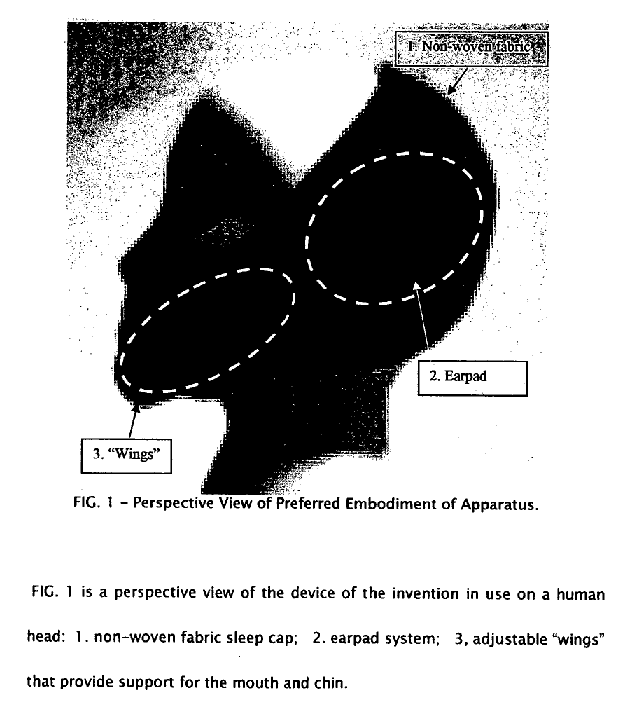

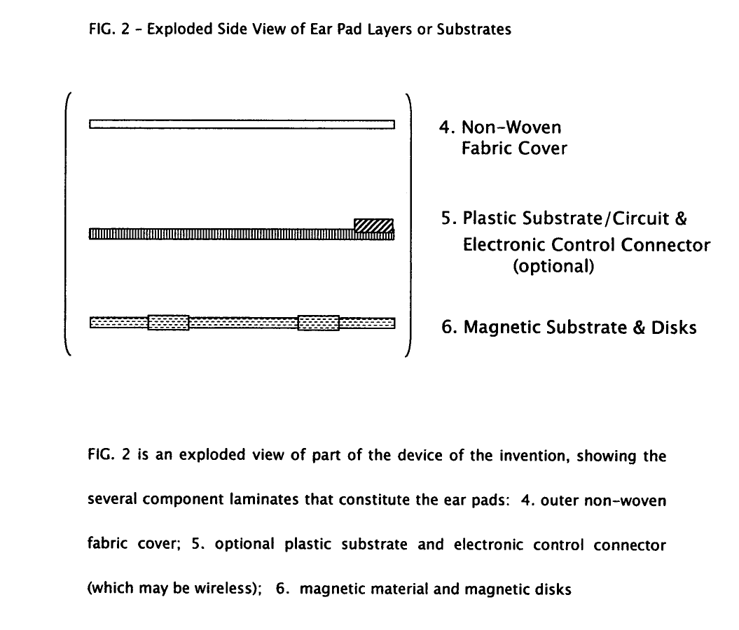

The present invention provides an improved method and apparatus for treatment of non-apnea snoring in adults, chronic snoring that is not directly the result of obstructive sleep apnea (OSA) or other sleep disorders as defined by the American Medical Association (AMA) and/or the Federal Drug Administration (FDA) where snoring is classified as a symptom. The method provides the method for snoring reduction or elimination through jaw and mouth support, acupressure, magnetic therapy, and optional electronically controlled very low voltage electromagnetic current applied to and around certain acupuncture points on the wearer's head. The preferred form of the invention is an apparatus that is designed to be worn much like a sleep cap by a snorer while sleeping to aid in reducing or eliminating snoring in adults. In a preferred form of the invention, the user wears an apparatus on the head during sleep which is comprised of a non-woven sleep cap with two embedded ear pads. The preferred form of the invention gently cradles and supports the mouth and chin, keeping it closed during sleep. This apparatus may reduce or eliminate non-apnea snoring in many adults.
1. Referring to FIG. 1, the device of the invention is located on the head of the user. The ear pads are self-positioned over the user's ears, shown as (1). The non-woven fabric that the device of the invention is made of provides an elastic head support feature which can be self-adjusted for fit and support, shown as (2). The elastic jaw, chin and mouth support features, called “wings,” can be adjusted for fit and support, shown as (3). The optional electronic control system connector is shown as (5).
2. Snoring is generally caused by air flowing through the open mouth, more than through the nasal passages. This causes turbulence and vibrates the soft tissue of the uvula and pharynx, resulting in snoring. The device of the invention keeps the sleeper's mouth gently closed and snoring is reduced or may even be eliminated in many sleepers.
3. The device of the present invention is shown in more detail in FIG. 2. It consists of an article of clothing constructed from non-woven fabric such that it resembles a fighter pilot's flight helmet liner. Embedded within the fabric (4) is a pair of composite ear pads constructed of a fabric layer (5) or similar material with a magnetic sheet substrate. An optional very low-voltage electro-magnetic circuit and stimulation control system (6), is described constructed from ceramic or neodymium magnetic material, laminated with the electrically conductive plastic and magnetic substrates, together with an adjustable elastic head support band. The optional 9-volt electrical stimulation and control subsystem may be useful in aiding the wearer through reduced snoring and better sleep.
4. The ear pads fit around and over the wearer's ears and are held in place by the fabric. Physical pressure is intermittently applied by the ear pads, head, and mouth support systems to certain acupuncture “points” on the wearer's head as the sleeper changes position during the night. Magnetic therapy is applied through the ear pads to certain acupuncture “points” around the ears and each side of the head as the earpads come in close proximity to these points. Very low-voltage electromagnetic stimulation may also be applied with the optional electronic control system to certain acupuncture “points” and contacts around the ears and on the face and neck.
5. The ear pads can be made of any suitable material. The outer “comfort” layer is constructed of a non-woven fabric material in a preferred embodiment. Where used, am optional thin plastic substrate between the outer non-woven fabric and the magnetic sheet material can be made of electrically conductive, coated Mylar or other suitable flexible and durable conductive plastic material. An optional low-voltage electrical circuit and contact points may be etched into the plastic material as required, and connected to an optional electronic control unit.
6. The magnetic substrate can be made of permanent magnetic sheet material which is magnetized with equally-spaced poles, normally either 10 or 14 poles per inch, running parallel to the length of the sheet. The anisotropic magnetic material can be of variable thickness, and normally has an energy level between 1.0 MGOe and 3.5 MGOe. Optionally, the magnetic sheet can be made of ceramic or neodymium material composed of strontium carbonate and iron oxide, with an energy level approximately 2.5-5.0 MGOe, which provides a stronger magnetic field within the composite structure of the ear pads, or other magnetic material suitable to the purpose. The device of the invention may also incorporate thin super strong magnets such as those based on iron (magnetic ferrite) or neodymium (particularly neodymium-iron-boron (NdFeB)) in order to provide super strong static magnetism points by which magneto-therapy may be provided. These magnets may be located in proximity to certain acupuncture “points” located around the ears and jaw, as well as elsewhere on the surface of the head and neck within the non-woven fabric device.
7. Because this preferred implementation of the invention provides for an optional electronic control feature, the use of more powerful and adjustable electromagnetic fields in lieu of or in addition to the magnetic sheet material is also included in the design. Such electromagnetic fields may be created via the use of a 9-volt DC current, the use of permanent magnetic material, or both. Control may be provided in both manual and automated manners by use of an optional control module that may be either physically attached to the device of the invention, or operated in a wireless manner.
8. The head and mouth support systems are nominally constructed of non-woven fabric with both elastic and comfort qualities (such as a cotton blend). They may also be constructed from woven fabrics using traditional machine sewn techniques. The earpads are embedded within the non-woven fabric, nominally by sandwiching them between two layers of non-woven fabric. Construction methods may include sewn or other fusing techniques, depending on the type(s) of non-woven fabrics used.

The embodiments of the invention in which an exclusive property or privilege is claimed are defined as follows:

1. A method of treating snoring in adult humans comprising:
a) adjustable support for the jaw, chin, and mouth to keep the mouth closed during sleep;
b) stimulation by means of applying physical pressure to and around certain acupuncture and acupressure “points” on the head;
c) application of both diffuse and focused magnetic fields that are applied on and around certain acupuncture and acupressure “points” on the head.
d) application of very small pulsed and/or persistent electrical current to the wearer's skin surface at and around acupuncture “points” through an optional 9-volt battery-powered electronic control system.

2. A lightweight, adjustable apparatus for keeping the mouth closed during sleep. Worn during sleep for reducing or eliminating non-apnea, mouth-related snoring in adults, the apparatus is comprised of:
a) an adjustable elastic device which embodies a bio-mechanical design that supports the jaw, chin and mouth, together with acupressure and magnetic therapy features, in such a manner as to gently keep the mouth firmly closed during sleep, thus reducing or temporarily stopping snoring caused by open-mouth breathing;
d) An ear pad system of two individual and non-identical composite pads that are constructed of suitable hypoallergenic or non-allergenic material, with one or more embedded substrates of magnetic materials;
e) An optional substrate of plastic material (which may be electrically conductive) which can contain a circuit and very low-voltage power source for enhanced stimulation of acupuncture and acupressure points on and around the ear;
f) The ear pad design incorporates gentle, and both intermittent and persistent pressure on and around certain acupressure points on the head (as many as 94 specific points per the World Health Organization (WHO) classification of acupuncture and acupressure proposed standards, as documented in the World Health Organization's Medical Library Proposed Standards for acupuncture and acupressure. These acupressure points may be beneficial in aiding in the reduction of stress, help induce sleep, and reduce or eliminate the tendency toward snoring in humans;
g) the ear pad may contain a composite magnetic substrate of both sheet and point magnetic materials that provides both focused and diffuse magnetic therapy that is applied to certain acupuncture areas by means of a combination of different magnetic materials and methods.
h) an embodiment of the ear pad may contain an electrically conductive plastic substrate that permits a very small pulsed and/or persistent electrical current to be applied through contacts to the wearer's skin surface on the head and neck at and around certain acupuncture “points” by an optional 9-volt battery-powered control system.

3. The method of claim 1 wherein the user places said apparatus on the head, adjusting both the position and tension of the fabric sleep cap and mouth support “wings,” as well as the position of each ear pad for comfort.

4. The method of claim 1 wherein the apparatus supports the mouth in a manner that permits the wearer to sleep with the mouth closed, reducing or eliminating most oral-related snoring.

5. The method of claim 1 wherein the wearer experiences concurrent stimulation through both persistent and intermittent physical pressure applied to certain acupuncture and acupressure points on the head and neck by action of the device being worn on the sleeper's head while asleep—the frequent movement of the sleeper creates these varying pressures.

6. The method of claim 1 wherein the wearer experiences concurrent stimulation through magnetic therapy applied to certain acupuncture and acupressure points on the head and neck.

7. The method of claim 1, wherein the wearer experiences a small pulsed and/or persistent electrical current through an etched circuit on an electrically-conductive substrate in the ear pad, and connected to both the magnetic substrate and contacts on the surface of the ear pad that penetrate the outer covering, which may be attached to an optional 9-volt DC battery powered electronic control system. This method can deliver a pulsed and/or persistent very low-voltage, very low-amperage electro-magnetic field at and near acupuncture points on the head and neck through optional surface contacts that connect the electrically-conductive and magnetic substrates and protrude through the foam outer covering next to the wearer's skin surface. The micro-current may also be conducted through contacts placed in the head and jaw support systems.

8. Device may also be constructed without the embedded earpad system; this method is also claimed in this patent. The resulting device can help keep the mouth closed during sleep, but does not offer the added features provided by the earpad system.
